(19) United States
(12) Patent Application Publication
(10) Pub. No.: US 2018/0104493 A1
(43) Pub. Date: Apr. 19, 2018

(54) METHODS TO PROGRAM SUB-PERCEPTION SPINAL CORD STIMULATION

(71) Applicant: Boston Scientific Neuromodulation Corporation, Valencia, CA (US)

(72) Inventors: Que T. Doan, West Hills, CA (US); Jianwen Gu, Valencia, CA (US)

(21) Appl. No.: 15/729,797

(22) Filed: Oct. 11, 2017

Related U.S. Application Data
(60) Provisional application No. 62/410,025, filed on Oct. 19, 2016.

(51) Int. Cl. A61N 1/36

(52) U.S. Cl. CPC A61N 1/36132; A61N 1/36071; A61N 1/36157; A61N 1/36171; A61N 1/36139

(57) ABSTRACT

This document discusses, among other things, systems and methods to provide a sub-perception spinal cord stimulation (SCS) to a patient. Some system examples include a stimulation device configured to deliver electrical stimulation to a patient and a control circuit configured to adjust at least one parameter of the electrical stimulation in response to a feedback signal from the patient.

### CLAIM OF PRIORITY

This application claims the benefit of priority under 35 U.S.C. § 119(e) of U.S. Provisional Patent Application Ser. No. 62/410,025, filed on Oct. 19, 2016, which is herein incorporated by reference in its entirety.

### TECHNICAL FIELD

This document relates generally to medical devices, and more particularly, to systems, devices and methods for delivering neural modulation.

### BACKGROUND

Neurostimulation, also referred to as neuromodulation, has been proposed as a therapy for a number of conditions. Examples of neurostimulation include Spinal Cord Stimulation (SCS), Deep Brain Stimulation (DBS), Peripheral Nerve Stimulation (PNS), and Functional Electrical Stimulation (FES). Implantable neurostimulation systems have been applied to deliver such a therapy. An implantable neurostimulation system may include an implantable neurostimulator, also referred to as an implantable pulse generator (IPG), and one or more implantable leads each including one or more electrodes. The implantable neurostimulator may deliver neurostimulation energy through one or more electrodes placed on or near a target site in the nervous system, and an external programming device may be used to program the implantable neurostimulator with stimulation parameters controlling the delivery of the neurostimulation energy.

Neurostimulation energy may be delivered using electrical energy that may have stimulation parameters to specify spatial (where to stimulate), temporal (when to stimulate) and/or informational (stimulation patterns directing the nervous system to respond as desired) aspects of a pattern of neurostimulation pulses.

### SUMMARY

An example (e.g., “Example 1”) of subject matter (e.g., a system) may include a stimulation means for delivering electrical stimulation to a spinal cord of a patient, the electrical stimulation having a first, second, and third parameter, and a control means for setting the first parameter of the electrical stimulation to a first value, setting the second parameter of the electrical stimulation to a second value, setting the third parameter of the electrical stimulation to a third value where the electrical stimulation is below a sensory threshold of the patient, increasing the value of the third parameter from the third value to increase the electrical stimulation until the electrical stimulation reaches the sensory threshold of the patient, storing the value of the third parameter corresponding to the sensory threshold of the patient in a storage, resetting the value of the second parameter of the electrical stimulation to a lower value below the second value, setting a value of the third parameter of the electrical stimulation to the stored value of the third parameter, increasing the value of the second parameter of the electrical stimulation from the lower value to increase the electrical stimulation until the electrical stimulation reaches the sensory threshold of the patient, and storing the value of the second parameter corresponding to the sensory threshold of the patient. In an example, the stimulation means may include a stimulation device, such as stimulation device 104 shown in FIG. 1, and at least one electrode, such as electrodes 106 shown in FIG. 1. In an example, the stimulation means may include a stimulation device, such as stimulation device 204 as shown in FIG. 2, and at least one lead system, such as lead system 208 as shown in FIG. 3. In an example, the control means may include a programming device, such as programming device 102 shown in FIG. 1. In an example, the control means may include a programming device, such as programming device 302 as shown in FIG. 3. In an example, the stimulation means may include a dedicated circuit or block of circuits including logic, or a set of machine readable instructions for causing a processor to perform as indicated and described with respect to blocks 104 and 106 of FIG. 1 or 204 and 208 of FIG. 2. In an example, the control means may include a dedicated circuit or block of circuits including logic, or a set of machine readable instructions for causing a processor to perform as indicated and described with respect to blocks 102 of FIG. 1 or 302 of FIG. 3, for example by causing an external device such as a programmer or patient remote control to communicate to an implantable pulse generator as needed to achieve the recited function(s).

In Example 2, the subject matter of Example 1 may optionally be configured such that the control means includes means for setting the value of the third parameter of the electrical stimulation to a value of a lower limit threshold if the stored value of the third parameter is less than the lower limit threshold, and set the value of the third parameter of the electrical stimulation to a value of an upper limit threshold if the stored value of the third parameter is greater than the upper limit threshold.

In Example 3, the subject matter of Example 2 may optionally be configured such that the control means may include means for setting the value of the second parameter to a value between forty and seventy percent of the value of the stored second parameter and increasing or decreasing the value of the second parameter based on a feedback signal from the patient at a time interval.

In Example 4, the subject matter of Example 3 may optionally be configured such that the feedback signal may be a physiological signal corresponding to at least one of a heart rate, a heart rate variability, a blood pressure, a respiration rate, or a skin conductance.

In Example 5, the subject matter of Example 3 may optionally be configured such that the feedback signal may be a neurological signal measured by at least one of electroencephalography or electromyography, or electric potentials sensed by the leads.

In Example 6, the subject matter of Example 3 may optionally be configured such that the feedback signal may be a functional parameter corresponding to at least one of an activity level, a posture, or a gait.

In Example 7 the subject matter of any one or any combination of Examples 1-6 may optionally be configured such that the first parameter may be a frequency, the second parameter may be an amplitude, and the third parameter may be a pulse width.

In Example 8 the subject matter of any one or any combination of Examples 2-7 may optionally be configured such that the sensory threshold of the patient may be determined based on the feedback signal.

In Example 9 the subject matter of any one or any combination of Examples 2-8 may optionally be configured such that the value of the lower limit threshold may be in a range of 10 μs to 50 μs.

In Example 10 the subject matter of any one or any combination of Examples 2-8 may optionally be configured such that the value of the lower limit threshold and the upper limit threshold may be in a range of 1 μs to 400 μs.

In Example 11 the subject matter of any one or any combination of Examples 2-8 may optionally be configured such that the value of the upper limit threshold may be in a range of 200 μs to 400 μs.

In Example 12 the subject matter of any one or any combination of Examples 7-11 may optionally be configured such that the first value is in a range of 1 kHz to 10 kHz.

In Example 13 the subject matter of any one or any combination of Examples 7-12 may optionally be configured such that the second value may be in a range of 3 mA to 5 mA.

In Example 14 the subject matter of any one or any combination of Examples 7-13 may optionally be configured such that the control means may include a means for increasing or decreasing the frequency based on the feedback signal at the time interval.

In Example 15 the subject matter of any one or any combination of Examples 7-14 may optionally be configured such that the stimulation means may include a means for delivering a spread bipole stimulation.

An Example, (e.g., “Example 16”) of subject matter (e.g., method) may include delivering electrical stimulation to a spinal cord of a patient, the electrical stimulation having a first, second, and third parameter, setting the first parameter of the electrical stimulation to a first value, setting the second parameter of the electrical stimulation to a second value, setting the third parameter of the electrical stimulation to a third value where the electrical stimulation is below a sensory threshold of the patient, increasing the value of the third parameter from the third value to increase the electrical stimulation until the electrical stimulation reaches the sensory threshold of the patient, storing the value of the third parameter corresponding to the sensory threshold of the patient in a storage, resetting the value of the second parameter of the electrical stimulation to a lower value below the second value, setting a value of the third parameter of the electrical stimulation to the stored value of the third parameter, increasing the value of the second parameter of the electrical stimulation from the lower value to increase the electrical stimulation until the electrical stimulation reaches the sensory threshold of the patient, and storing the value of the second parameter corresponding to the sensory threshold of the patient.

In Example 17 the subject matter of Example 16 may optionally include setting the value of the third parameter of the electrical stimulation to a value of a lower limit threshold if the stored value of the third parameter is less than the lower limit threshold, and setting the third parameter of the electrical stimulation to a value of an upper limit threshold if the stored value of the third parameter is greater than the upper limit threshold.

In Example 18 the subject matter of Example 17 may optionally include setting the second parameter to a value between forty and seventy percent of the value of the stored second parameter, and increasing or decreasing the second parameter based on a feedback signal from the patient at a time interval.

In Example 19 the subject matter of Example 18 may optionally be configured such that the feedback signal may be a physiological signal corresponding to at least one of a heart rate, a heart rate variability, a blood pressure, a respiration rate, and a skin conductance.

In Example 20 the subject matter of Example 18 may optionally be configured such that the feedback signal may be a neurological signal measured by at least one of electroencephalography, electromyography, or electric potentials sensed by the leads.

In Example 21 the subject matter of Example 18 may optionally be configured such that the feedback signal may be a functional parameter corresponding to at least one of an activity level, a posture, or a gait.

In Example 22 the subject matter of Example 18 may optionally be configured such that the first parameter may be a frequency, the second parameter is an amplitude, and the third parameter is a pulse width.

In Example 23 the subject matter of Example 17 may optionally be configured such that the sensory threshold of the patient may be determined based on the feedback signal from the patient.

In Example 24 the subject matter of Example 17 may optionally be configured such that the value of the lower limit threshold may be in a range of 1 μs to 400 μs.

In Example 25 the subject matter of Example 17 may optionally be configured such that the value of the lower limit threshold may be in a range of 10 μs to 50 μs.

In Example 26 the subject matter of Example 17 may optionally be configured such that the value of the upper limit threshold may be in a range of 1 μs to 400 μs.

In Example 27 the subject matter of Example 17 may optionally be configured such that the value of the upper limit threshold may be in a range of 200 μs to 400 μs.

In Example 28 the subject matter of Example 22 may optionally be configured such that the first value may be in a range of 1 kHz to 10 kHz.

In Example 29 the subject matter of Example 22 may optionally be configured such that the second value may be in a range of 3 mA to 5 mA.

In Example 30 the subject matter of Example 22 may optionally include increasing or decreasing the frequency based on the feedback signal from the patient at the time interval.

In Example 31 the subject matter of Example 22 may optionally be configured such that the delivering the electrical stimulation to the spinal cord of the patient may include delivering a spread bipole stimulation to the spinal cord of the patient.

An example, (e.g., “Example 32”) of subject matter (e.g., a method) may include delivering sub-perception spinal cord stimulation (SCS) having a power level to a patient, the delivered sub-perception SCS providing pain relief to the patient, and adjusting a value of at least one parameter of the delivered sub-perception SCS to reduce the power level of the delivered SCS while maintaining the pain relief to the patient.

In Example 33 the subject matter of Example 32 may optionally be configured such that at least one parameter includes at least one of a frequency, an amplitude, or a pulse width.

In Example 34 the subject matter of Example 32 may optionally be configured such that delivering sub-perception SCS includes delivering sub-perception SCS to a physical midline of the patient.

In Example 35 the subject matter of Example 32 may optionally be configured such that delivering sub-perception SCS includes using an implanted bipole to deliver the sub-perception SCS.

An example (e.g., “Example 36”) of subject matter (e.g., system) may include a stimulation device configured to deliver electrical stimulation to a patient, and a control circuit configured to adjust at least one parameter of the electrical stimulation in response to a feedback signal.

In Example 37 the subject matter of Example 36 may optionally be configured such that the feedback signal includes a physiological signal including at least one of a heart rate, blood pressure, respiration rate, and skin conductance.

In Example 38 the subject matter of Example 36 may optionally be configured such that the feedback signal includes a neurological signal measured by at least one of electroencephalography or electromyography.

In Example 39 the subject matter of Example 36 may optionally be configured such that the feedback signal includes a functional parameter including at least one of activity level, posture, or gait.

In Example 40 the subject matter of any one or any combination of Examples 36-39 may optionally be configured such that the stimulation device may be configured to deliver electrical stimulation to a spinal cord of a patient, the electrical stimulation having a first, second, and third parameter, and the control circuit may be configured to set a value of the first parameter of the electrical stimulation to a first value, set a value of the second parameter of the electrical stimulation to a second value, set a value of the third parameter of the electrical stimulation to a third value where the electrical stimulation may be below a sensory threshold of the patient, increase the value of the third parameter from the third value to increase the electrical stimulation until the electrical stimulation reaches the sensory threshold of the patient, store the value of the third parameter corresponding to the sensory threshold of the patient in a storage, reset the value of the second parameter of the electrical stimulation to a lower value below the second value, set a value of the third parameter of the electrical stimulation to the stored value of the third parameter, increase the value of the second parameter of the electrical stimulation from the lower value to increase the electrical stimulation until the electrical stimulation reaches the sensory threshold of the patient, and store the value of the second parameter corresponding to a sensory threshold of the patient.

In Example 41 the subject matter of Example 40 may optionally be configured such that the control circuit may be further configured to set the value of the third parameter of the electrical stimulation to a value of a lower limit threshold if the stored value of the third parameter may be less than the lower limit threshold, and set the value of the third parameter of the electrical stimulation to a value of an upper limit threshold if the stored value of the third parameter may be greater than the upper limit threshold.

In Example 42 the subject matter of Example 41 may optionally be configured such that the control circuit may be further configured to set the value of the second parameter to a value between forty and seventy percent of the value of the stored second parameter, and increase or decrease the second parameter based on a feedback signal at a time interval.

In Example 43 the subject matter of any one or any combination of Examples 40-42 may optionally be configured such that the first parameter may be a frequency, the second parameter may be an amplitude, and the third parameter may be a pulse width.

In Example 44 the subject matter of Example 41 may optionally be configured such that the sensory threshold of the patient may be determined based on the feedback signal.

In Example 45 the subject matter of Example 43 may optionally be configured such that the value of the lower limit threshold and the upper limit threshold are in a range of 1 μs to 400 μs.

In Example 46 the subject matter of Example 43 may optionally be configured such that the first value may be in a range of 1 kHz to 10 kHz.

In Example 47 the subject matter of Example 43 may optionally be configured such that the second value may be in a range of 2 mA to 6 mA.

In Example 48 the subject matter of Example 43 may optionally be configured such that the second value may be in a range of 0.1 mA to 6 mA.

In Example 49 the subject matter of Example 43 may optionally be configured such that the control circuit may be further configured to increase or decrease the frequency based on the feedback signal at the time interval.

In Example 50 the subject matter of any one or any combination of Examples 36-49 may optionally be configured such that the stimulation device may be configured to deliver sub-perception spinal cord stimulation (SCS) therapeutically effective to provide pain relief, the SCS having a power level, and the control circuit may be operatively connected to the stimulation device to adjust the power level of the SCS to a reduced power level while maintaining therapeutic effectiveness in providing pain relief.

An example (e.g., “Example 51”) of subject matter (e.g., a system or apparatus) may optionally combine any portion or combination of any portion of any one or more of Examples 1-50 to include “means for” performing any portion of any one or more of the functions or methods of Examples 1-50, or a “machine-readable medium” (e.g., a non-transitory medium or a medium having a mass, etc.) including instructions that, when performed by a machine, cause the machine to perform any portion of any one or more of the functions or methods of Examples 1-50.

An example (e.g., “Example 52”) of subject matter (e.g., a system) may include a stimulation device for delivering electrical stimulation to a spinal cord of a patient, the electrical stimulation having a first, second, and third parameter, and a programming device for setting the first parameter of the electrical stimulation to a first value, setting the second parameter of the electrical stimulation to a second value, setting the third parameter of the electrical stimulation to a third value where the electrical stimulation is below a sensory threshold of the patient, increasing the value of the third parameter from the third value to increase the electrical stimulation until the electrical stimulation reaches the sensory threshold of the patient, storing the value of the third parameter corresponding to the sensory threshold of the patient in a storage, resetting the value of the second parameter of the electrical stimulation to a lower value below the second value, setting a value of the third parameter of the electrical stimulation to the stored value of the third parameter, increasing the value of the second parameter of the electrical stimulation from the lower value to increase the electrical stimulation until the electrical stimulation reaches the sensory threshold of the patient, and storing the value of the second parameter corresponding to the sensory threshold of the patient.

In Example 53, the subject matter of Example 52 may optionally be configured such that the programming device may set the value of the third parameter of the electrical stimulation to a value of a lower limit threshold if the stored value of the third parameter is less than the lower limit threshold, and set the value of the third parameter of the electrical stimulation to a value of an upper limit threshold if the stored value of the third parameter is greater than the upper limit threshold.

In Example 54, the subject matter of Example 53 may optionally be configured such that the programming device may set the value of the second parameter to a value between forty and seventy percent of the value of the stored second parameter and increase or decrease the value of the second parameter based on a feedback signal from the patient at a time interval.

In Example 55, the subject matter of Example 54 may optionally be configured such that the feedback signal may be a physiological signal corresponding to at least one of a heart rate, a heart rate variability, a blood pressure, a respiration rate, or a skin conductance.

In Example 56, the subject matter of Example 54 may optionally be configured such that the feedback signal may be a neurological signal measured by at least one of electroencephalography or electromyography, or electric potentials sensed by the leads.

In Example 57, the subject matter of Example 54 may optionally be configured such that the feedback signal may be a functional parameter corresponding to at least one of an activity level, a posture, or a gait.

In Example 58 the subject matter of any one or any combination of Examples 52-57 may optionally be configured such that the first parameter may be a frequency, the second parameter may be an amplitude, and the third parameter may be a pulse width.

In Example 59 the subject matter of any one or any combination of Examples 53-58 may optionally be configured such that the sensory threshold of the patient may be determined based on the feedback signal.

In Example 60 the subject matter of any one or any combination of Examples 53-59 may optionally be configured such that the value of the lower limit threshold may be in a range of 10 μs to 50 μs.

In Example 61 the subject matter of any one or any combination of Examples 53-59 may optionally be configured such that the value of the lower limit threshold and the upper limit threshold may be in a range of 1 μs to 400 μs.

In Example 62 the subject matter of any one or any combination of Examples 53-59 may optionally be configured such that the value of the upper limit threshold may be in a range of 200 μs to 400 μs.

In Example 63 the subject matter of any one or any combination of Examples 58-62 may optionally be configured such that the first value is in a range of 1 kHz to 10 kHz.

In Example 64 the subject matter of any one or any combination of Examples 58-63 may optionally be configured such that the second value may be in a range of 3 mA to 5 mA.

In Example 65 the subject matter of any one or any combination of Examples 58-64 may optionally be configured such that the programming device may increase or decrease the frequency based on the feedback signal at the time interval.

In Example 66 the subject matter of any one or any combination of Examples 58-65 may optionally be configured such that the stimulation device may deliver a spread bipole stimulation.

This summary is intended to provide an overview of subject matter of the present patent application. It is not intended to provide an exclusive or exhaustive explanation of the disclosure. The detailed description is included to provide further information about the present patent application. Other aspects of the disclosure will be apparent to persons skilled in the art upon reading and understanding the following detailed description and viewing the drawings that form a part thereof, each of which are not to be taken in a limiting sense.

### BRIEF DESCRIPTION OF THE DRAWINGS

Various embodiments are illustrated by way of example in the figures of the accompanying drawings. Such embodiments are demonstrative and not intended to be exhaustive or exclusive embodiments of the present subject matter.

FIG. 1 illustrates an example of a neurostimulation system.

FIG. 2 illustrates an example of a stimulation device and a lead system.

FIG. 3 illustrates an example of a programming device.

FIG. 4 illustrates an example of an implantable neurostimulation system.

FIG. 5 illustrates an example of an implantable stimulator and one or more leads of an implantable neurostimulation system.

FIG. 6 illustrates an example of an external programming device of an implantable neurostimulation system.

FIG. 7 illustrates an example of an implantable pulse generator (IPG) and percutaneous leads.

FIG. 8 illustrates an example for operating a neurostimulation system.

FIG. 9 illustrates an example for operating a neurostimulation system.

FIG. 10 illustrates an example for operating a neurostimulation system.

### DETAILED DESCRIPTION

Various examples provide a method of adjusting parameters of a spinal cord stimulation therapy such as to provide pain relief to a patient.

Current neurostimulation systems may be programmed to deliver waveforms having parameters such as a frequency, amplitude, and pulse width. In a sub-threshold spinal cord stimulation (SCS) system, pain relief may develop after a latent period ranging from hours to days. To determine a set of parameters that provide pain relief, a clinician may set the waveform parameters to experimentally predetermined values. After waiting for a time greater than or equal to the latent period, the patient may provide feedback to the clinician to determine if the delivered waveform is providing pain relief. The clinician may then repeatedly adjust the waveform parameters while waiting for a time greater than or equal to the latent period between each adjustment. Various examples disclosed herein provide a method for optimizing the waveform parameters in a sub-threshold SCS stimulation system.

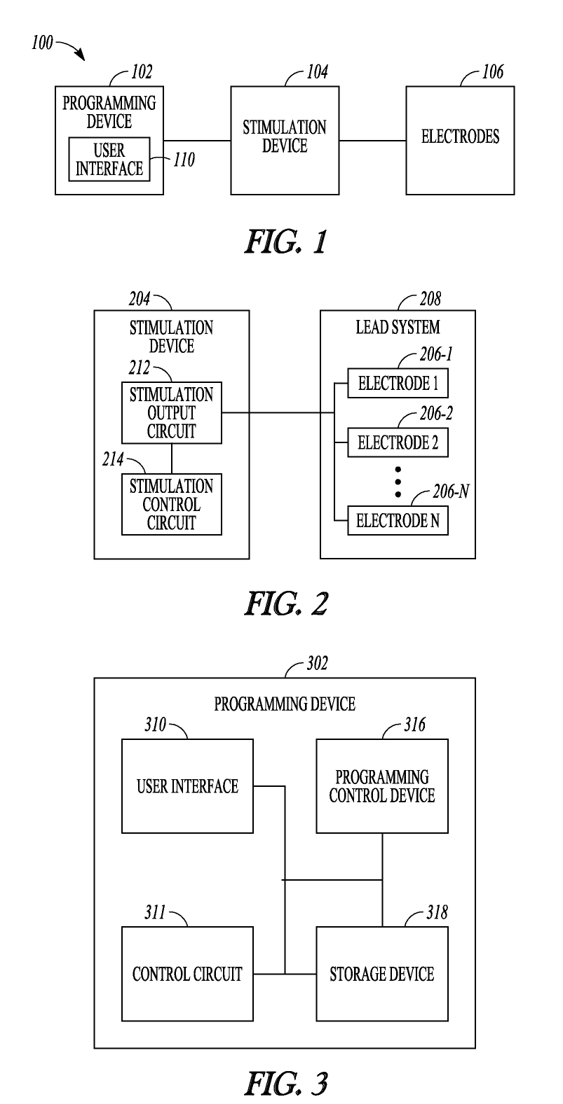

FIG. 1 illustrates, by way of example and not limitation, an embodiment of a neurostimulation system 100. The neurostimulation system 100 may include electrodes 106, a stimulation device 104, and a programming device 102. Electrodes 106 may be configured to be placed on or near one or more neural targets in a patient. Stimulation device 104 may be configured to be electrically connected to electrodes 106 and deliver neurostimulation energy, such as in the form of electrical pulses, to the one or more neural targets though electrodes 106. The delivery of the neurostimulation may be controlled using a plurality of stimulation parameters, such as stimulation parameters specifying a waveform shape such as, but not limited to, a pattern of the electrical pulses and a selection of electrodes through which each of the electrical pulses is delivered. At least some parameters of the plurality of stimulation parameters may be programmable by a user, such as a physician or other caregiver who treats the patient using system 100. Programming device 102 may provide the user with accessibility to the user-programmable parameters. The programming device 102 may be configured to be communicatively coupled to stimulation device 104 via a wired or wireless link. The programming device 102 may receive a feedback signal from the patient and based on the feedback signal, the programming device 102 may automatically adjust the stimulation parameters, such as to provide improved pain relief to the patient.

In an example, programming device 102 includes a user interface that allows the user to set and/or adjust values of the user-programmable parameters by creating and/or editing graphical representations of various waveforms. Such waveforms may include different waveform shapes. The waveform shapes may include regular shapes (e.g. square, sinusoidal, triangular, saw tooth, and the like) or irregular shapes. The waveform shapes may include regular or irregular patterns. The waveform shapes may be similar to analog signals or may be similar to digitized signals. By way of example and not limitation, the waveforms may include a pattern of temporal waveform segments, which may include a pattern of neurostimulation pulses, to be delivered to the patient. In an example, the

FIG. 2 illustrates, by way of example and not limitation, an embodiment of a stimulation device 204 and a lead system 208, such as may be implemented in neurostimulation system 100. The stimulation device 204 may include a stimulation output circuit 212 and a stimulation control circuit 214. Stimulation output circuit 212 may produce and deliver neurostimulation pulses. Stimulation control circuit 214 may control the delivery of the neurostimulation pulses using the plurality of stimulation parameters, which specifies a pattern of the neurostimulation pulses. Lead system 208 may include one or more leads each configured to be electrically connected to stimulation device 204 and a plurality of electrodes 206 distributed in the one or more leads. The plurality of electrodes 206 may include electrode 206-1, electrode 206-2, . . . electrode 206-N, each a single electrically conductive contact providing for an electrical interface between stimulation output circuit 212 and tissue of the patient, where N≥2. The neurostimulation pulses may be delivered from stimulation output circuit 212 through a set of electrodes selected from electrodes 206.

The number of leads and the number of electrodes on each lead may depend on, for example, the distribution of target(s) of the neurostimulation and the need for controlling the distribution of electric field at each target. By way of example and not limitation, lead system 208 may include two leads each having eight electrodes. Other lead systems, such as but not limited to, four lead systems or paddle leads may be used.

FIG. 3 illustrates, by way of example and not limitation, an embodiment of a programming device 302, such as may be implemented in neurostimulation system 100. The programming device 302 may include a storage device 318, a control circuit 311, a programming control circuit 316, and a user interface 310. Storage device 318 may store a plurality of temporal waveform segments. The temporal waveform segments may include pulses and may include other waveform shapes. Programming control circuit 316 may generate the plurality of stimulation parameters that control the delivery of the neurostimulation according to the pattern of the neurostimulation pulses. The control circuit 311 may receive a feedback signal and may adjust the plurality of stimulation parameters based on the received feedback signal. The feedback signal may be a physiological signal, a neurological signal, or a functional parameter. The physiological signal may be a heart rate, heart rate variability, blood pressure, respiration rate, or skin conductance. The neurological signal may be measured by electroencephalography or electromyography, or electric potentials sensed by the leads. The functional parameter may include an activity level, posture, or gait. The control circuit may adjust at least one of a pulse width, amplitude, and frequency of the neurostimulation in response to the measured feedback signal. User interface 310 may allow the user to select or compose a pattern of neurostimulation pulses. User interface 310 may allow the user to select an automatic mode where the neurostimulation parameters are automatically updated in response to a received feedback signal.

In an example, user interface 310 may include, but is not limited to, a touchscreen. For example, user interface 310 may include any type of presentation device, such as interactive or non-interactive screens, and any type of user input devices that allow the user to edit the waveforms and schedule a program, such as touchscreen, keyboard, keypad, touchpad, trackball, joystick, and mouse. A program may receive feedback from the patient at a time interval and adjust the stimulation parameters in response to the feedback. The circuits of neurostimulation system 100, including its various embodiments discussed in this document, may be implemented using a combination of hardware and software. For example, the circuit of user interface 110 or 310, stimulation control circuit 214, and programming control circuit 316, including their various embodiments discussed in this document, may be implemented using an application-specific circuit constructed to perform one or more particular functions or a general-purpose circuit programmed to perform such function(s). Such a general-purpose circuit may include, but is not limited to, a microprocessor or a portion thereof, a microcontroller or portions thereof, and a programmable logic circuit or a portion thereof.

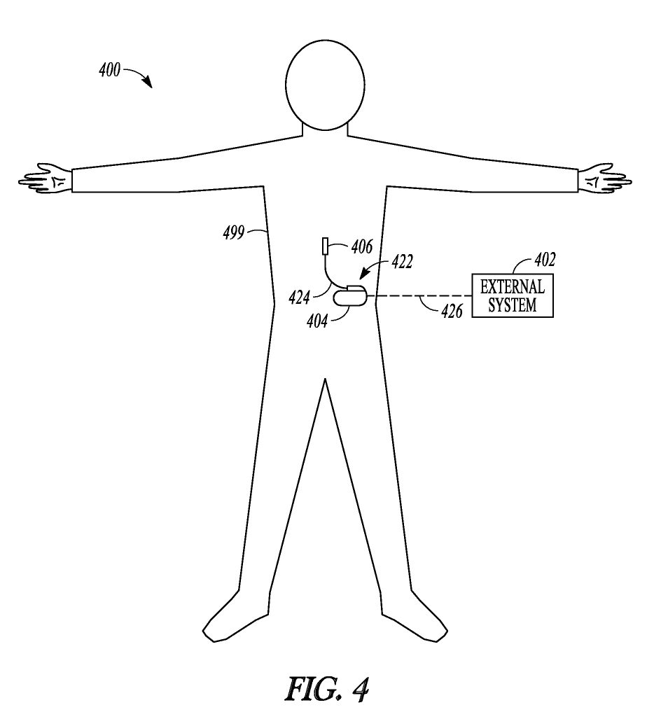

FIG. 4 illustrates, by way of example and not limitation, an implantable neurostimulation system 400 and portions of an environment in which system 400 may be used. The system 400 may include an implantable system 422, an external system 402, and a telemetry link 426 providing for wireless communication between implantable system 422 and external system 402. In an example, implantable system 422 is illustrated in FIG. 4 as being implanted in the patient's body 499.

The implantable system 422 may include an implantable stimulator (also referred to as an implantable pulse generator, or IPG) 404, a lead system 424, and electrodes 406, which represent an embodiment of stimulation device 204, lead system 208, and electrodes 206, respectively. The external system 402 may represent an example of programming device 302. The external system 402 may include one or more external (non-implantable) devices each allowing the user and/or the patient to communicate with implantable system 422. The external system 402 may include a programming device intended for the user to initialize and adjust settings for implantable stimulator 404 and a remote control device intended for use by the patient. For example, the remote control device may allow the patient to turn implantable stimulator 404 on and off and/or adjust certain patient-programmable parameters of the plurality of stimulation parameters. The external system 402 may include a programming device configured to automatically initialize settings for the implantable stimulator 404 and then based on a received feedback signal (e.g., a physiological signal, a neurological signal, or a functional parameter) from the patient, automatically adjust the settings for the implantable stimulator 404, such as to provide pain relief to the patient.

The sizes and shapes of the elements of implantable system 422 and their location in body 499 are illustrated by way of example and not by way of restriction. In various examples, the present subject matter may be applied in programming any type of stimulation device that uses electrical pulses as stimuli, regardless of stimulation targets in the patient's body and whether the stimulation device is implantable.

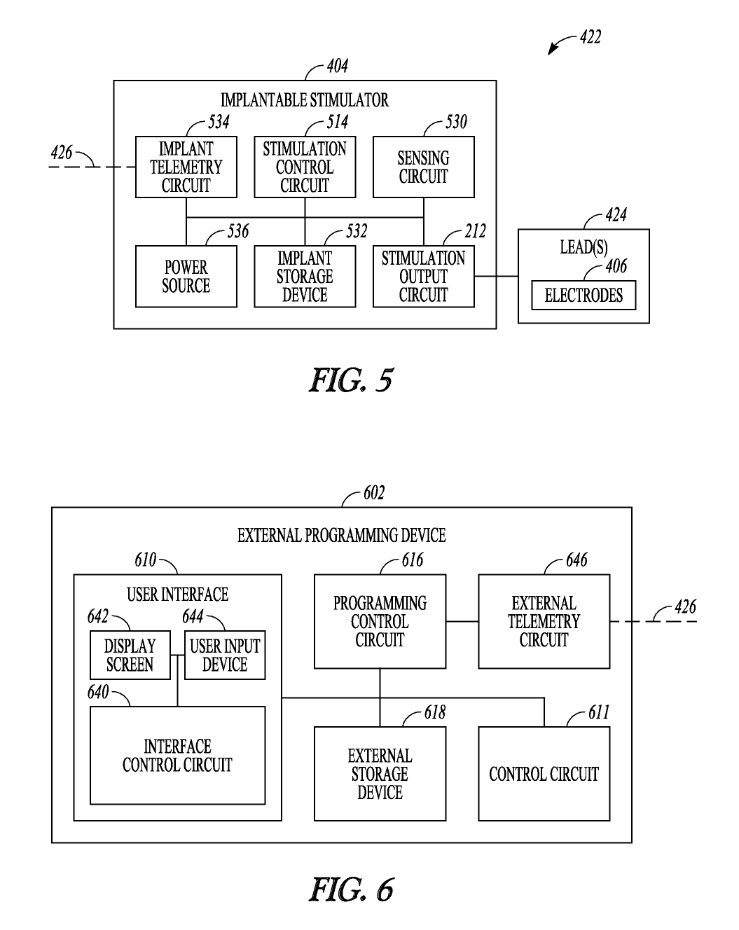

FIG. 5 illustrates, by way of example and not limitation, an embodiment of implantable stimulator 404 and one or more leads 424 of an implantable neurostimulation system, such as implantable system 422. The implantable stimulator 404 may include a sensing circuit 530 that is optional and required only when the stimulator has a sensing capability, stimulation output circuit 212, a stimulation control circuit 514, an implant storage device 532, an implant telemetry circuit 534, and a power source 536. The sensing circuit 530 may sense one or more physiological signals for purposes of patient monitoring and/or feedback control of the neurostimulation. The physiological signals may include neural and other signals each indicative of a condition of the patient that is treated by the neurostimulation and/or a response of the patient to the delivery of the neurostimulation. The sensing circuit 530 may sense a signal such as a physiological signal, a neurological signal, or a functional parameter. By way of example and not limitation, the physiological signal may be a heart rate, heart rate variability, blood pressure, respiration rate, or skin conductance. By way of example and not limitation, the neurological signal may be measured by electroencephalography or electromyography, or electric potentials sensed by the leads. By way of example and not limitation, the functional parameter may include an activity level, posture, or gait. The sensing circuit 530 can provide the sensed signal to the control circuit 611 and the control circuit 611 may adjust at least one of a pulse width, amplitude, and frequency of the neurostimulation in response to the sensed feedback signal to provide pain relief to the patient.

Stimulation output circuit 212 may be electrically connected to electrodes 406 through lead 424, and may deliver the neurostimulation through a set of electrodes selected from electrodes 406. Stimulation control circuit 514 may represent an example of stimulation control circuit 214 and may control the delivery of the neurostimulation using the plurality of stimulation parameters specifying the pattern of the neurostimulation. For example, stimulation control circuit 514 may control the delivery of the neurostimulation using the one or more sensed physiological signals. Implant telemetry circuit 534 may provide implantable stimulator 404 with wireless communication with another device such as a device of external system 402, including receiving values of the plurality of stimulation parameters from external system 402. Implant storage device 532 may store values of the plurality of stimulation parameters. Power source 536 may provide implantable stimulator 404 with energy for its operation. The power source 536 may include a battery, such as a rechargeable battery and a battery charging circuit for charging the rechargeable battery. Implant telemetry circuit 534 may also function as a power receiver that receives power transmitted from external system 402 through an inductive couple.

In various examples, sensing circuit 530, stimulation output circuit 212, stimulation control circuit 514, implant telemetry circuit 534, implant storage device 532, and power source 536 are encapsulated in a hermetically sealed implantable housing. In various examples, lead(s) 424 are implanted such that electrodes 406 are placed on and/or around one or more targets to which the neurostimulation pulses are to be delivered, while implantable stimulator 404 is subcutaneously implanted and connected to lead(s) 424 at the time of implantation.

FIG. 6 illustrates, by way of example and not limitation, an embodiment of an external programming device 602 of an implantable neurostimulation system, such as external system 402. External programming device 602 may represent an example of programming device 302, and may include an external telemetry circuit 646, an external storage device 618, a programming control circuit 616, a control circuit 611, and a user interface 610.

In an example, external telemetry circuit 646 provides external programming device 602 with wireless communication with another device such as implantable stimulator 404 via telemetry link 426, including transmitting the plurality of stimulation parameters to implantable stimulator 404. The external telemetry circuit 646 may also transmit power to implantable stimulator 404 through the inductive couple.

The external storage device 618 may store a plurality of temporal waveform segments selectable for use as a temporal segment of the neurostimulation waveform. Examples of such waveforms include pulses, bursts each including a group of the pulses, trains each including a group of the bursts, and sequences each including a group of the pulses, bursts, and trains. External storage device 618 may also store a plurality of stimulation fields. Each field of the plurality of stimulation fields may be defined by one or more electrodes of the plurality of electrodes through which a pulse of the neurostimulation pulses is delivered and a current distribution of the pulse over the one or more electrodes.

The programming control circuit 616 may generate a plurality of stimulation parameters, which are to be transmitted to implantable stimulator 404, according to the pattern of the neurostimulation pulses. In various examples, programming control circuit 616 checks values of the plurality of stimulation parameters against safety rules to limit these values within constraints of the safety rules. The safety rules may be heuristic rules.

The user interface 610 may allow the user to define the pattern of neurostimulation pulses and perform various other monitoring and programming tasks. The user interface 610 may allow the user to select a feedback based pattern of neurostimulation wherein the feedback based pattern of neurostimulation may include an initial neurostimulation pattern that may be subsequently modified based on a feedback signal received from a patient. The user interface 610 may include a GUI. User interface 610 may include a display screen 642, a user input device 644, and an interface control circuit 640. Display screen 642 may include any type of interactive or non-interactive screens, and user input device 644 may include any type of user input devices that supports the various functions discussed in this document, such as touchscreen, keyboard, keypad, touchpad, trackball, joystick, and mouse. The interface control circuit 640 may control the operation of user interface 610 including responding to various inputs received by user input device 644 and defining the one or more stimulation waveforms.

The external programming device 602 may have operation modes including a composition mode and a real-time programming mode. In the composition mode (also known as the pulse pattern composition mode), user interface 610 may be activated, while programming control circuit 616 may be inactivated. In an example, programming control circuit 616 does not dynamically update values of the plurality of stimulation parameters in response to any change in the one or more stimulation waveforms. In the real-time programming mode, both user interface 610 and programming control circuit 616 may be activated. Programming control circuit 616 may dynamically update values of the plurality of stimulation parameters in response to changes in the set of one or more stimulation waveforms, and transmit the plurality of stimulation parameters with the updated values to implantable stimulator 404.

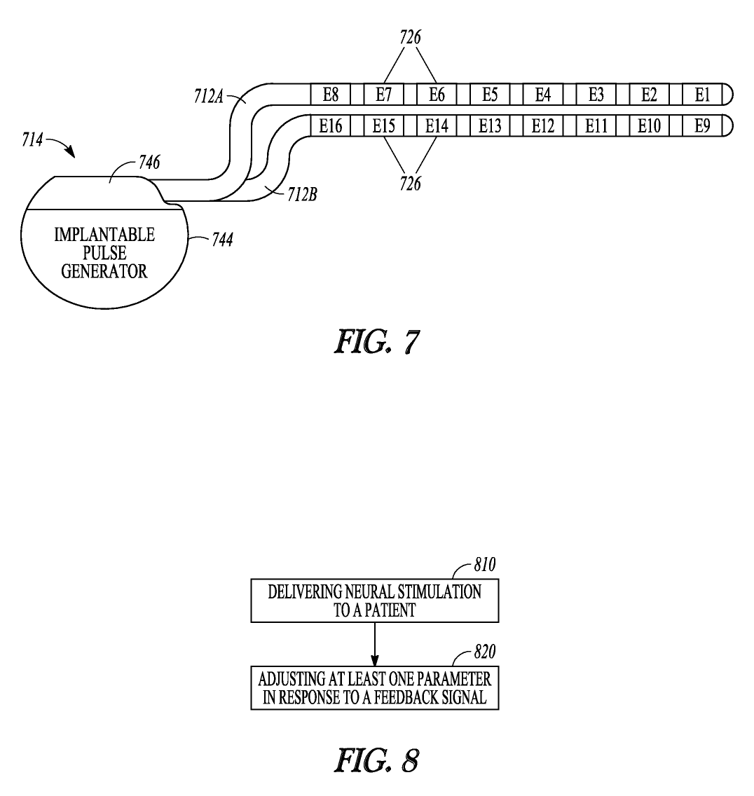

FIG. 7 illustrates, by way of example and not limitation, an example of a profile view of an implantable pulse generator (IPG) 744 and percutaneous leads 712. One of the neuromodulation leads 712a may have eight electrodes 726 (labeled E1-E8), and the other neuromodulation lead 712b may have eight electrodes 726 (labeled E9-E16). The actual number and shape of leads and electrodes may, of course, vary according to the intended application. The IPG 14 may comprise an outer case 744 for housing the electronic and other components (described in further detail below), and a connector 746 to which the proximal ends of the neuromodulation leads 712 mates in a manner that electrically couples the electrodes 726 to the electronics within the outer case 744. The outer case 744 may be composed of an electrically conductive, biocompatible material, such as titanium, and forms a hermetically sealed compartment wherein the internal electronics are protected from the body tissue and fluids. In some examples, the outer case 744 may serve as an electrode.

In an example, the IPG 714 includes a battery and pulse generation circuitry that delivers the electrical modulation energy in the form of one or more electrical pulse trains to the electrode array 726 in accordance with a set of modulation parameters programmed into the IPG 714. Such modulation parameters may comprise electrode combinations, which define the electrodes that are activated as anodes (positive), cathodes (negative), and turned off (zero), percentage of modulation energy assigned to each electrode (fractionalized electrode configurations), and electrical pulse parameters that may define the pulse amplitude (which may be measured in milliamps or volts depending on whether the IPG 714 supplies constant current or constant voltage to the electrode array 726), pulse duration (which may be measured in microseconds), pulse rate (which may be measured in pulses per second), and burst rate (which may be measured as the modulation on duration X and modulation off duration Y).

In an example, electrical modulation may occur between two (or more) activated electrodes, one of which may be the IPG case 744. Modulation energy may be transmitted to the tissue in a monopolar or multipolar (e.g., bipolar, tripolar, etc.) fashion. Monopolar modulation may occur when a selected one of the lead electrodes 726 is activated along with the case of the IPG 714, so that modulation energy is transmitted between the selected electrode 726 and case. Bipolar modulation may occur when two of the lead electrodes 726 are activated as anode and cathode, so that modulation energy is transmitted between the selected electrodes 726. For example, electrode E3 on the first lead 712a may be activated as an anode at the same time that electrode E11 on the second lead 712a is activated as a cathode. Tripolar modulation may occur when three of the lead electrodes 726 are activated, two as anodes and the remaining one as a cathode, or two as cathodes and the remaining one as an anode. For example, electrodes E4 and E5 on the first lead 712a may be activated as anodes at the same time that electrode E12 on the second lead 712b is activated as a cathode. The modulation energy may be delivered between a specified group of electrodes as monophasic electrical energy or multiphasic electrical energy.

FIG. 8 illustrates an example for operating a neurostimulation system, such as neurostimulation system 100, such as to provide pain relief to a patient. The neurostimulation system 100 may deliver stimulation to a patient (step 810). The neurostimulation system 100 may include a control circuit, such as control circuit 311 that may be configured to receive a feedback signal (e.g., physiological signal, neurological signal, or functional parameter) and adjust at least one parameter (e.g., pulse width, amplitude, or frequency) of the delivered stimulation in response to the received feedback signal (step 820).

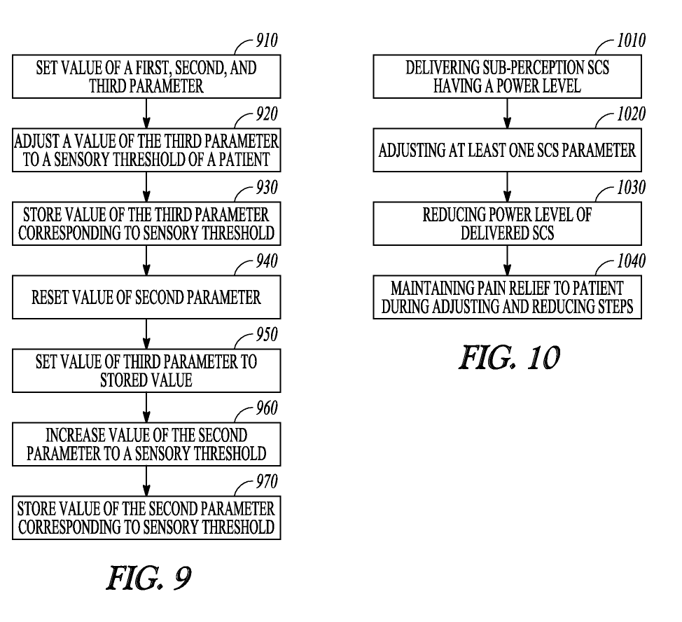

FIG. 9 illustrates, by way of example and not limitation, a method for operating a neurostimulation system, such as neurostimulation system 100. The illustrated method may be used to deliver a pain relief therapy. The pain relief therapy may include delivering sub-threshold spinal cord stimulation (SCS) to a patient. In trial-and-error testing, the adjustment of parameters of the delivered SCS may take a relatively long time. The illustrated method may provide an improvement over trial-and-error methods of determining parameters of the delivered SCS where wash-in and wash-out times may be relatively long (e.g., hours or days). The neurostimulation system 100 may deliver electrical stimulation to a spinal cord of the patient. The electrical stimulation may include a first parameter, a second parameter, and a third parameter. The first parameter may be a frequency, the second parameter may be an amplitude, and the third parameter may be a pulse width. The frequency may be set to a value in the range of 1 kHz to 10 kHz. The amplitude may be set to a value in the range of 2 mA to 6 mA. The amplitude may be set to a value in the range of 0.1 mA to 6 mA. The amplitude may be set to a value in the range of 3 mA to 5 mA. A control circuit, such as control circuit 311 may set a value of the first parameter to a first value, a value of the second parameter to a second value, and a value of the third parameter to a third value where the electrical stimulation is below a sensory threshold of the patient (step 910). The control circuit 311 may set a frequency of the electrical stimulation to a value in the range of 1 to 10 kHz, an amplitude of the electrical stimulation to 5 mA, and a pulse width to a value below a sensory threshold of the patient. A control circuit, such as control circuit 311 may increase the value of the third parameter until the electrical stimulation reaches the sensory threshold of the patient (step 920). The pulse width may then be increased until the electrical stimulation is above the sensory threshold of the patient. The value of the third parameter corresponding to the sensory threshold of the patient may be stored, such as in the storage device 318 (step 930). The value of the pulse width corresponding to the sensory threshold of the patient may then be stored. A control circuit, such as control circuit 311 may reset the value of the second parameter of the electrical stimulation to a lower value below the second value (step 940). The amplitude of the electrical stimulation may be reduced to 0 mA. A control circuit, such as control circuit 311 may set a value of the third parameter to the stored value of the third parameter (step 950). The pulse width may be set to the stored value. A control circuit, such as control circuit 311 may increase the value of the second parameter until the electrical stimulation reaches the sensory threshold of the patient (step 960). The amplitude of the electrical stimulation may be increased until the electrical stimulation is above the sensory threshold of the patient. The value of the second parameter corresponding to a sensory threshold of the patient may be stored, such as in storage device 318 (step 970). The value of the amplitude corresponding to the sensory threshold of the patient may be stored. The value of the third parameter of the electrical stimulation may be set to a value of a lower limit threshold if the stored value of the third parameter is less than the lower limit threshold and the value of the third parameter of the electrical stimulation may be set to a value of an upper limit threshold if the stored value of the third parameter is greater than the upper limit threshold. The third parameter may be a pulse width and the lower limit threshold may be in a range of 1 μs to 400 μs. The lower limit threshold may be in a range of 10 μs to 50 μs. The lower limit threshold may be in a range of 200 μs to 400 μs. The value of the second parameter may be set to a value between forty and seventy percent of the value of the stored second parameter; and a value of the second parameter may be increased or decreased based on a feedback signal at a time interval.

FIG. 10 illustrates, by way of example and not limitation, a method for operating a neurostimulation system, such as neurostimulation system 100, such as to provide pain relief to a patient. The illustrated method may be used to deliver a pain relief therapy. In certain systems, a relatively low power level is used to provide spinal cord stimulation (SCS). Initially, the patient may feel no pain relief and the parameters of the SCS may then be adjusted. After waiting for a wash-in time, the patient may be queried to determine if the adjusted parameters are providing pain relief. Further adjustments may be made until parameters are determined that provide pain relief to the patient. Unfortunately, in such a system, the patient may not experience pain relief while the parameters are being adjusted. The illustrated method may provide the patient with pain relief during the adjustment of parameters by utilizing a relatively high power to provide SCS. The power level can be reduced as parameters are adjusted such as to provide pain relief to the patient during the determination of optimal SCS parameters.

The neurostimulation system 100 may deliver sub-perception spinal cord stimulation (SCS) having a power level to a patient (step 1010). The power level may be selected such that pain relief is provided to the patient. At least one parameter of the delivered SCS may then be adjusted, such as to reduce the power level of the delivered SCS while maintaining pain relief to the patient. The at least one parameter may be a frequency, amplitude, or pulse width. The delivered SCS may be delivered to a spinal cord of the patient. The sub-perception SCS may be delivered by an implanted bipole or other electrode configurations.

Various embodiments are illustrated in the figures above. One or more features from one or more of these embodiments may be combined to form other embodiments.

Method examples described herein can be machine or computer-implemented at least in part. Some examples may include a computer-readable medium or machine-readable medium encoded with instructions operable to configure an electronic device or system to perform methods as described in the above examples. An implementation of such methods can include code, such as microcode, assembly language code, a higher-level language code, or the like. Such code can include computer readable instructions for performing various methods. The code can form portions of computer program products. Further, the code can be tangibly stored on one or more volatile or non-volatile computer-readable media during execution or at other times.

The above detailed description is intended to be illustrative, and not restrictive. The scope of the disclosure should, therefore, be determined with references to the appended claims, along with the full scope of equivalents to which such claims are entitled.

What is claimed is:

1. A method comprising:
delivering electrical stimulation to a spinal cord of a patient, the electrical stimulation having a first, second, and third parameter;
setting the first parameter of the electrical stimulation to a first value;
setting the second parameter of the electrical stimulation to a second value;
setting the third parameter of the electrical stimulation to a third value where the electrical stimulation is below a sensory threshold of the patient,
increasing the value of the third parameter from the third value to increase the electrical stimulation until the electrical stimulation reaches the sensory threshold of the patient;
storing the value of the third parameter corresponding to the sensory threshold of the patient in a storage;
resetting the value of the second parameter of the electrical stimulation to a lower value below the second value;
setting a value of the third parameter of the electrical stimulation to the stored value of the third parameter;
increasing the value of the second parameter of the electrical stimulation from the lower value to increase the electrical stimulation until the electrical stimulation reaches the sensory threshold of the patient; and
storing the value of the second parameter corresponding to the sensory threshold of the patient.

2. The method of claim 1 further comprising:
setting the value of the third parameter of the electrical stimulation to a value of a lower limit threshold if the stored value of the third parameter is less than the lower limit threshold; and
setting the third parameter of the electrical stimulation to a value of an upper limit threshold if the stored value of the third parameter is greater than the upper limit threshold.

3. The method of claim 2 further comprising;
setting the second parameter to a value between forty and seventy percent of the value of the stored second parameter; and
increasing or decreasing the second parameter based on a feedback signal from the patient at a time interval.

4. The method of claim 3 wherein the feedback signal is a physiological signal corresponding to at least one of a heart rate, a heart rate variability, a blood pressure, a respiration rate, or a skin conductance.

5. The method of claim 3 wherein the feedback signal is a neurological signal measured by at least one of electroencephalography, electromyography, or electric potentials sensed by the leads.

6. The method of claim 3 wherein the feedback signal is a functional parameter corresponding to at least one of an activity level, a posture, or a gait.

7. The method of claim 3 wherein the first parameter is a frequency, the second parameter is an amplitude, and the third parameter is a pulse width.

8. The method of claim 2 wherein the sensory threshold of the patient is determined based on the feedback signal from the patient.

9. The method of claim 2 wherein the value of the lower limit threshold is in a range of 1 μs to 400 μs.

10. The method of claim 2 wherein the value of the lower limit threshold is in a range of 10 μs to 50 μs.

11. The method of claim 2 wherein the value of the upper limit threshold is in a range of 1 μs to 400 μs.

12. The method of claim 2 wherein the value of the upper limit threshold is in a range of 200 μs to 400 μs.

13. The method of claim 7 wherein the first value is in a range of 1 kHz to 10 kHz.

14. The method of claim 7 wherein the second value is in a range of 3 mA to 5 mA.

15. The method of claim 7 further comprising increasing or decreasing the frequency based on the feedback signal from the patient at the time interval.

16. The method of claim 7 wherein the delivering the electrical stimulation to the spinal cord of the patient includes delivering a spread bipole stimulation to the spinal cord of the patient.

17. A method comprising:
delivering sub-perception spinal cord stimulation (SCS) having a power level to a patient, the delivered sub-perception SCS providing pain relief to the patient; and
adjusting a value of at least one parameter of the delivered sub-perception SCS to reduce the power level of the delivered sub-perception SCS while maintaining the pain relief to the patient.

18. The method of claim 17 wherein the at least one parameter includes at least one of a frequency, an amplitude, or a pulse width.

19. The method of claim 17, wherein delivering sub-perception SCS includes delivering sub-perception SCS to a physical midline of the patient.

20. The method of claim 17, wherein delivering sub-perception SCS includes using an implanted bipole to deliver the sub-perception SCS.
